(19) United States
(12) Patent Application Publication
(10) Pub. No.: US 2007/0259435 A1
(43) Pub. Date: Nov. 8, 2007

(54) Intravenous Drug Administration and Blood Sampling Model in the Awake Rat

(75) Inventors: Claire Elisabeth Mackie, Antwerpen (BE); Marc Jozef Adolphine Haseldonckx, Hoogstraten (BE); Saskia Sabine Blokland, Breda (NL); Koen Wuyts, Retie (BE); Petra Carla Gysemberg, Herentals (BE); Iris Juliana Martha Verhoeven, Willebroek (BE); Philip Maria Martha Timmerman, Hasselt (BE); Maria Johanna Magdalena Aldina Nijsen, Beerse (BE)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Janssen Pharmaceutica N.V., Turnhoutseweg 30 (BE)

(21) Appl. No.: 11/597,119

(22) PCT Filed: May 24, 2005
(86) PCT No.: PCT/EP05/52381

§ 371 Date: Nov. 21, 2006

(30) Foreign Application Priority Data
May 28, 2004 (EP) 04102381.3

(51) Int. Cl. G01N 33/48

(52) U.S. Cl. 436/63

(57) ABSTRACT

There is a continuing need for increased throughput in the examination of new chemical entities (NCEs) in terms of the pharmacokinetic (PK) parameters. The aim was to validate a new study method which allows a higher throughput, the examination of inter-animal variability and a reduction in the numbers of animals needed for routine bioavailability studies of NCEs in awake rats. The design uses a new method for intravenous (iv) administration via the saphenous vein in combination with serial blood sampling via the tail vein. The multiple sampling method was compared with single sampling (decapitation) and the effect on haematocrit (Hct) levels was studied. Direct injection in the saphenous vein was compared to iv administration using an indwelling jugular catheter. Using structural different CE's, it was shown that a combination of direct injection via the saphenous vein and multiple sampling from the tail vein produces comparable plasma concentrations and subsequent PK results to the comparator methods. Furthermore, Hct levels remained within recommended levels using a total blood sampling volume of up to 2.1 ml per day. The new technique increases throughput by reducing the time required for preparative surgery, increases the quality by allowing inter-animal comparison of major PK parameters as concentration time curves can be collected from each animal and reduces the number of animals required.

### FIELD OF THE INVENTION

The present invention relates to a new intravenous drug administration and blood sampling model in the awake rat, comprising at least the steps of (a) intravenous administration of a chemical entity through the saphenous vein and (b) sampling the blood from the tail vein.

### BACKGROUND OF THE INVENTION

The pharmaceutical industry as a whole is acutely aware of the development time and costs incurred to deliver a new chemical entity (NCE) to the market. Within the Drug Discovery ADME (Absorption Distribution Metabolism Excretion) field there are many different ways of investigating the early drug-like properties of chemical entities (CE)s. Two major areas are those involving in vitro assay systems investigating one/two parameters or end points and in vivo models using the whole animal system. Until now much of the effort has been focused on the development of high-throughput in vitro metabolism and absorption assays (Bajpai, M.; Adkison, K. K. Curr. Opin. Drug Discovery Dev. 2000, 3, 63-71) and increasing speed of analytical capabilities in bioanalytical assays (Cox, K. A.; White, R. E.; Korfmacher, W. A. Comb. Chem. High Throughput. Screen. 2002, 5, 29-37). As part of the Drug Discovery effort there is a continuing need for an increased throughput in the examination of CEs in terms of the in vivo pharmacokinetic (PK) parameters. To obtain these parameters and bioavailability of an CE, the compound is dosed via the intravenous (iv) and oral (po) routes, blood is sampled at various time points and then analysed by e.g. liquid chromatography coupled to tandem mass spectrometry (LC-MS/MS) for the compound of interest. The PK parameters (e.g. clearance, volume of distribution, elimination half-life and oral bioavailability), which describe the absorption and disposition in the whole animal, are then calculated from the plasma concentration time profile. Efforts to increase throughput in in vivo PK evaluation of CEs have focused on mixture dosing and sample pooling to minimize bioanalytical workload (Allen, M. C.; Shah, T. S.; Day, W. W. Pharm. Res. 1998, 15, 93-97; Olah, T. V.; McLoughlin, D. A.; Gilbert, J. D. Rapid Commun. Mass Spectrom. 1997, 11, 17-23 ; Hop, C. E.; Wang, Z.; Chen, Q.; Kwei, G. J. Pharm. Sci. 1998, 87, 901-903 and Shaffer, J. E.; Adkison, K. K.; Halm, K.; Hedeen, K.; Berman, J. J. Pharm. Sci. 1999, 88, 313-318). These methods however have associated disadvantages. Cassette dosing can increase the possibility of adverse effects and drug-drug interactions in the animal and compromise bioanalysis.

With the post-dose pooling of plasma samples bioanalysis can also be compromised due to sample dilution and additional method development time to prevent undue co-elution and subsequent ion suppression in the LC-MS/MS.

### DESCRIPTION OF THE INVENTION

The aim of the present invention was to increase the throughput of the in life or in vivo part of routine rat PK studies by designing and validating an analytical method being a combination approach of a new iv administration route using the saphenous vein and blood sampling, in particular multiple blood sampling via the tail vein in the awake rat. This analytical method can also comprise appropriate bioanalytical techniques to allow the processing/analysis of the blood/plasma samples and estimation of the major PK parameters.

The invention therefore relates to an analytical method for the determination of a pharmakokinetic parameter in the awake rat, comprising the subsequent steps of:

(a) intravenous administration of a chemical entity through the saphenous vein;

(b) sampling the blood from the tail vein.

In the context of this application, a CE is any compound or chemical, either from natural or synthetic origin, such as, but not limited thereto, pharmaceuticals, active compounds, vitamins, proteins and viruses.

### Saphenous Vein Administration

In the context of this application, the saphenous vein is a vein located at the surface of the hind limb to drain away blood from the hind limb.

Several methods for the in-life phase (dose administration and blood sampling) of rat PK studies have been employed across the industry and as with all methods each has its own advantages and disadvantages. With respect to the route of iv dosing and sampling, prior art describes:

1) iv administration and sampling respectively to and from the tail vein; advantage is that no preparative surgery is required, but blood samples could be contaminated at early sampling time points due to residue dose solution remaining at the site of administration. Also if one vein does not sample well the other lateral vein can become damaged due to over sampling;

2) tail vein administration and sampling via the orbital plexus; advantage is that dosing and sampling is from two discrete sites and no preparative surgery is required, but the animal must be anaesthetised prior to blood withdrawal. Each animal should only undergo this type of procedure for a limited number of times within a 24 h period. Several animals must be used to obtain sufficient blood samples at the required time points to be able to construct an adequate concentration time profile and PK parameters;

3) tail vein administration and serial blood sampling from the carotid/jugular vein; advantage is that dosing and sampling is from two discrete sites and that small frequent blood samples can be removed within the same animal for adequate description of the plasma concentration time profile, but preparative surgery is required The same arguments remain true for the combination of iv administration via an indwelling jugular cannula and sampling from the tail vein;

4) iv administration and blood sampling from the same indwelling jugular catheter; advantage is that small frequent blood samples can be removed within the same animal for adequate description of the plasma concentration time profile, but blood samples at early time-points could be contaminated due to residue dosing solutions remaining in the cannula and also preparative surgery is required.

5) iv administration through the saphenous vein and sampling the blood from the tail vein of the anaesthetised rat (EP 1 284 139 A1, published 19 Feb. 2003). The difference with the current application is the fact that the rat is awake instead of anaesthetised. Although this difference may seem small, the method according to the invention has never been published before, despite the massive amount of work that is carried out and published in this scientific area. Clearly, the invention has overcome a technical prejudice, and has further provided o.a. the advantage that the interaction between the chemical entity administered and the anaesthetic and/or narcotic drug, such as, e.g. sodium pentobarbital, has been eliminated. It is without saying that also other parameters that could be influenced by the administration of the anaesthetic and/or narcotic drug are not influenced in the method according to the invention, especially in the case of the testing of CNS-drugs, such as, e.g. anti-anxiolytics, anti-psychotics, anti-depressives and anti-pain drugs. It also further provides the advantage that samples can be drawn at much faster rate from the same rat and without disturbing e.g. the rat's metabolism, as the rat does not need to wake up after being anaesthetized.

It was one aspect of the present invention to design and validate a new iv administration route, via direct injection of the CE into the saphenous vein in the awake rat. This method a.o. reduces the time required for preparative surgery and recovery (1-2 days). CEs with different chemical structures (n=11) were administered via the jugular or saphenous vein. Blood was sampled at various time points using the multiple blood sampling technique via the tail vein (see further), plasma samples were analysed for the appropriate CE and the major PK parameters were compared between the two iv routes.

It is noted that the saphenous vein is known in the prior art for blood sampling (A. Hem, A. J. Smith and P. Solberg Laboratory Animals 1998, 32, 364-368).

### Multiple Blood Sampling

With respect to the blood sampling procedure, there are several prior art methods for collecting samples at the required time points. One possibility is that the CE is administered to a set of animals (e.g. n=3), which is sacrificed at the appropriate time point for blood collection (by decapitation). An advantage is that large blood samples can be obtained, however the use of LC/MS/MS today allows for very small samples to be collected and analysed. The plasma concentration levels derived from this method are obtained from blood samples of a set of multiple animals, each sampled at different time points. Therefore, the data analysis and calculation of the major PK parameters can only be performed on the mean plasma concentration time profile. This method of sampling therefore does not allow the study of inter-individual variation in PK results. A major disadvantage is the high amount of animals needed for the study.

It was a further aspect of the present invention to investigate a new sampling regimen, in which a blood sample, in particular multiple blood samples were withdrawn from the tail vein at different time points from the same rat. The method of multiple blood sampling allows adequate concentration time profiles to be obtained from individual animals, permitting the calculation of the major PK parameters from each animal. In this way, inter-animal variability can be examined and the number of animals required to conduct a routine bioavailability study for one CE can be reduced significantly. CEs with different chemical structures were administered orally in rats and blood was withdrawn by single blood sampling (decapitation) or by multiple sampling from the tail vein at various time points. Plasma samples were analysed for the appropriate CE and the plasma concentration time profiles and calculated PK parameters were compared between the two sampling techniques. Sufficient blood samples in both number and volume are required from each rat to be able to construct a suitable plasma concentration time profile, to perform an appropriate extraction procedure and subsequent analyses by LC-MS/MS, but without affecting the well being of the animal. Therefore, the impact of multiple sampling on haematological parameters, such as the haematocrit (Hct), was investigated following blood removal at different volumes over the desired period of time.

Hence, the invention further relates to an analytical method for the determination of a pharmakokinetic parameter in the awake rat, wherein in step (b) multiple blood samples are taken from the tail vein.

The invention further relates to an analytical method for the determination of a pharmakokinetic parameter in the awake rat, wherein the method further comprises a step (c), incorporated after step (b) wherein the blood/plasma sample is analyzed using a bioanalytical technique to determine a pharmakokinetic parameter.

### Experimental

### 1. Materials And Methods

1.1. Animals

Male SPF Sprague-Dawley rats (200-300 g, Charles River, Germany) were used throughout the study. Animals were allowed to acclimatize for one week prior to each part of the study. Tap water and food were available ad libitum.

1.2. Validation of the Multiple Blood Sampling Technique

For multiple blood sampling via the tail vein the animals were placed in a rodent cylindrical restrainer developed by the Scientific Instrument Division (Johnson & Johnson, Pharmaceutical Research and Development, division of Janssen Pharmaceutica). The rat's tail was warmed using an infra red lamp to facilitate the sampling. Venous blood was collected by a 27 G needle into Multivette 600KE tubes containing EDTA (Sarstedt, Germany).

1.2.1. Influence of Multiple Blood Sampling On the Haematocrit Levels

The influence of the volume of blood removed over the required sampling period on rat Hct levels was investigated (n=5 per group). Multiple (7×) blood sampling volumes of 0.3 ml (total blood volume 2.1 ml) and 0.4 ml (total blood volume 2.8 ml) were compared. Blood was withdrawn at 7 and 20 min, 1, 2, 4, 8 and 24 h.

The % volume of the Hct was calculated using a diagnostic instrument (Advia 120, Bayer Diagnostics, Brussel, Belgium), which analyses whole blood to count white and red blood cells, platelets and reticulocytes.

1.2.2. Pharmacokinetic Validation of the Multiple Sampling Method

The plasma concentration profile and major PK parameters of different chemical structures were investigated following oral administration. Blood samples were taken from the tail vein with either the multiple or single sampling method.

1.2.2.1. Test Compounds And Formulations

For both methods, the same formulation per compound was used to ensure any differences in results were not due to formulation effects. Test compounds JNJ1 (a farnesyl transferase compound), JNJ2 (galantamine hydrobromide) and JNJ3 (a CRF antagonist) were formulated in demineralized water or a 10% hydroxypropyl- -β cyclodextrin (HP- - [symbol] CD) solution at final concentrations of between 0.25 and 1 mg/ml. All formulations were stored at room temperature, protected from light and analysed quantitatively. Animals were orally dosed by gastric intubation using a volume of 10 ml/kg.

1.2.2.2. Blood Sampling

After dosing the compound, blood samples were taken at the desired time points. For the single sampling method, three animals per time point were sacrificed by decapitation and blood was collected by exsanguination into 10 ml B-D Sterile EDTA K3 Vacutainer tubes. For the multiple sampling technique 0.3 ml venous blood was repeatedly collected from the tail vein as described above. For each compound, 3 rats were used for a complete plasma concentration time profile. Plasma samples were analysed for the appropriate compound using individual qualified research LC-MS/MS methods as described in section 2.4.

1.3. Comparison of Plasma PK Parameters Following Administration Via the Jugular Or Saphenous Vein

The plasma concentration profiles and major PK parameters of compounds covering different disease targets and chemical classes/structures (n=11, not all reported here) were compared following administration via a jugular vein (catheter) or the saphenous vein (direct injection) in the awake rat.

1.3.1. Test Compounds And Formulations

For both iv administration routes, the same formulation per compound was used to ensure any differences in results were not due to formulation effects. Compounds (n=11; among which is JNJ4 (an antipsychotic having 5HT₂-antagonism, D2-antagonism and SSRI activity), JNJ5 (an NK₁₂₃-antagonist) and JNJ6 (an HSD11β, antagonist)) were formulated as either aqueous solutions or HP-β-CD solutions (10-20%, pH range of 4 to 7) at a final concentration of 1.25 mg/ml. All formulations were made isotonic with mannitol, stored at room temperature, protected from light and analysed quantitatively. For all compounds, formulations were dosed at 2 ml/kg for both iv administration routes.

1.3.2. Intravenous Administration Route

1.3.2.1. Iv Administration Via the Jugular Vein

Indwelling catheters were placed into the jugular vein under general anaesthesia. Surgery was performed under sterile conditions; all surfaces on which surgery was carried out were covered with sterile absorbent impermeable surgical table drapes (Unidrape, Vygon, France), surgical instruments were sterilized in a 1/10 hibitane (5%)/alcohol (70%) mixture and surgery was performed with sterile surgical powder free gloves (NuTex®, Ansell Medical, Malaysia). Induction of anaesthesia and tracheal intubation was performed under 4% isoflurane (Forene®; Abbott, England) in a 30/70 O₂N₂O mixture. The rats were maintained under general anaesthesia using 1.5% isoflurane in a 30/70 O₂/N₂O mixture. An iv catheter was constructed from Silastic® Laboratory tubing (4 cm, ID 0.64 mm OD 1.19 mm; Dow Corning, USA) and polyethylene tubing (4 cm, PE 50; ID 0.58 mm and OD 0.965 mm; Becton Dickinson, Belgium). The tubes were fixed together with Loctite 404 industrial adhesive, (Loctite, USA). The catheter was inserted into the jugular vein and advanced into the subclavian vein to allow a good blood withdrawal. The position of the catheter was verified by drawing blood back into the cannula and then flushing with heparinised saline (100 I.U./ml; Heparin Leo, Belgium). The catheter was held in position using two sutures. The cannula was refilled with heparinised saline to keep it patent. The cannula was tunnelled subcutaneously and externalised through a small incision in the scruff of the neck by using a large needle. The end of the cannula was closed with a removable plug of steel. All wounds were closed using sterile suture (Mersilk® 4/0, Ethicon®, Belgium). All animals were allowed to recover for 48 hours in single cages before dose administration and had access to food and tap water ad libitum. After dosing (n=3 for each compound) the formulation the catheter was flushed with 0.1 ml saline.

1.3.2.2. Iv Administration Via the Saphenous Vein

An awake rat (n=3 for each compound) was given a direct injection via the saphenous vein with a needle (Microlance™, 27G3/4, 0.4×19, Becton Dickinson, Ireland) connected to a polyethylene tube PE 10 (ID 0.28 mm and OD 0.61 mm; Becton Dickinson, Belgium). Prior to administration the leg was shaved and the saphenous vein was slightly pinched on the proximal side to become more visible The rat was restrained with a towel during dose administration.

1.3.2.3. Blood Sampling

Individual blood samples (0.3 ml/time point; total blood volume 2.1 ml) were collected by the multiple sampling technique at 7 and 20 min, 1, 2, 4, 8 and 24 h after dosing. For each compound and for each route of administration, 3 to 5 rats were used for a complete plasma concentration time profile. Plasma samples were analysed for the appropriate compound using individual qualified research LC-MS/MS methods as described below. The experimental protocols adhere to the “Principles of Laboratory Animal Care published by the NIH (1985).

1.4. Bioanalysis

1.4.1. Method Development

Bioanalytical method validation according to the FDA (Guidance for Industry, Bioanalytical method validation, US department of Health and Human Services, Food and Drug Administration, CDER, 2001) is not a requirement in discovery, early developmental or mechanistic PK studies. Nevertheless, it is essential to use analytical methods that can provide plasma concentrations with sufficient accuracy and precision to allow valid decisions. For that purpose, qualified research LC-MS/MS methods were developed for each compound. The resulting methods showed similar accuracy and precision to the FDA standards, expressed by the statistics of the calibration curve and independent quality control samples analysed together with every set of study samples. In addition, limited plasma stability (2 h at 37° C.) was investigated to cover all manipulations of the plasma samples between sampling of the animals and analysis. Inter assay accuracy and precision was not investigated.

1.4.2. Sample Preparation

For all compounds, individual calibration curves were constructed in blank EDTA rat plasma (covering the expected concentration range). Independent quality control samples (QC) were prepared in rat EDTA plasma at four concentration levels in duplicate, covering the entire calibration range. The study samples, calibration and QC samples were extracted using a generic sample preparation method. After sample preparation, the (reconstituted) residues were pipetted in polypropylene auto sampler vials and analysed using individual qualified research methods.

1.4.3. LC-MS/MS

Aliquots of the residues were analysed using multiple reaction monitoring (MRM) liquid chromatography coupled to tandem mass spectrometry (LC-MS/MS) (API-3000 or 4000, Applied Biosystems, Canada). Chromatographic separation was obtained on commercial base deactivated C-18 columns of different brands, depending on the chromatographic behaviour of the compounds to be analysed. The mass spectrometers were operated in the TurbolonSpray TM mode (positive ion electrospray ionisation). For each compound the mass spectrometer was optimised to measure a selective parent-daughter transition. Integration of the chromatographic peaks was performed using the Analist software version 1.2 (Applied Biosystem, Canada). Final concentrations were calculated by interpolation from the calibration curves response, using a linear regression model. Both accuracy and precision were within expectations for all compounds, allowing accurate PK calculations

1.5. Data Analysis

A limited PK analysis was performed using WinNonlin™ Professional (Version 3.3). For the single sampling method, a non-compartmental data analysis was carried out on the mean plasma concentration profile (n=3/time point). For the multiple sampling method, a non-compartmental data analysis was carried out on the plasma concentration time profiles obtained from each animal (n=3). Average values were then calculated to compare the multiple and the single sampling method. The PK parameters calculated were observed maximum plasma concentration (Cₘₐₓ), the time to reach the maximum plasma concentration (Tₘₐₓ), plasma half-life (t_{1/2}) and exposure of the compound calculated by the area under the curve (AUCₗₐₛₜ and AUC_{inf}). For the comparison of the injection routes (jugular vein cannula and saphenous vein) the major parameters of half-life (t_{1/2}), volume of distribution (Vdₛₛ (compartmental analysis) or Vd_{z} (non-compartmental analysis)), total plasma clearance (Cl) and area under the curve (AUCₗₐₛₜ and AUC_{inf}) were determined for each individual animal to allow inter-animal comparisons.

### 2. Results

2.1. Validation of the Multiple Blood Sampling Technique

2.1.1. Influence of Multiple Blood Sampling On Haematocrit Levels

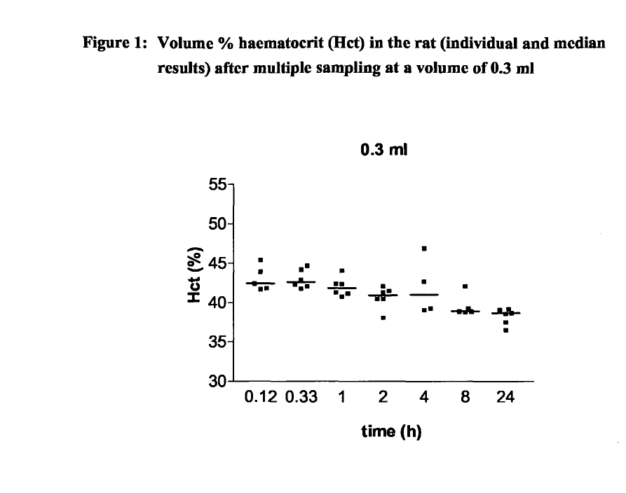

FIG. 1 shows the volume % haematocrit (Hct) in the rat (n=6) after multiple sampling at a volume of 0.3 ml. The initial blood samples showed Hct values ranging from 42 to 45 volume %, with a median of 42%. The median Hct value slightly decreased, resulting in a value of 39% at the last sampling point (at t=24 h). A one-way ANOVA and post hoc Student's t-test revealed that Hct levels were significantly (p<0.05) decreased as compared to the initial time-point after the 5^{th} blood sample (at t=8 and 24 h). However, these levels (ranging from 37 to 42%) were still within the range of expected values for the healthy rat (36-48%) (Havenaar, R.; Ritskes-Hoitinga, J.; Meijer, J. C.; Zwart, P. Biologie en zootechniek. In Proefdieren En Dierproeven; van Zutphen, L. F. M., Baumans, V., Beynen, A. C., Eds.; Wetenschappelijke uitgeverij Bunge: Utrecht, 1991; pp. 20-74).

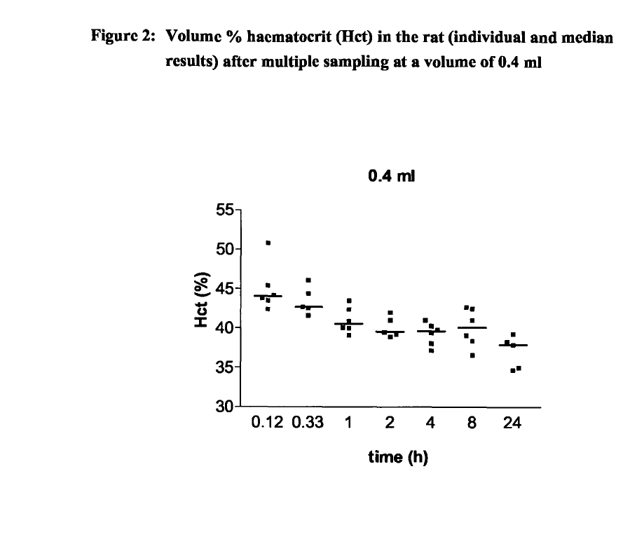

FIG. 2 shows the volume % Hct in the rat (n=6) after multiple sampling at a volume of 0.4 ml. The initial blood samples showed Hct values ranging from 42 to 51 volume %, with a median of 44%. The median Hct value slightly decreased, resulting in a value of 38% at the last sampling point (at t=24 h). A one-way ANOVA and post hoc Student's t-test revealed that Hct levels were significantly decreased as compared to the initial time-point after the 2^{nd} blood sample (at t=1, 2, 4, 8 and 24 h). At t=24 h these levels ranged from 35 to 39%.

2.1.2. Pharmacokinetic Validation of the Multiple Sampling Method

The individual or mean basic PK parameters following single or multiple blood sampling after oral administration of individual compounds (hereafter called JNJ1, JNJ2 and JNJ3) are shown in tables 1-3. As can be seen from the results both sampling techniques produced comparable PK parameters for the three compounds shown. Using JNJ1 as an example, mean maximum plasma concentrations (Cₘₐₓ) reached 63.6±17.5 ng/ml at 1±1 h for the multiple sampling group versus 41.4 ng/ml at 1 h for the single sampling group. The half-life (t_{1/2}) was 2.02±0.3 h for the multiple sampling group compared to 1.92 h for the single sampling group. The exposure as measured by AUC_{inf} was 294±46 ng.h/ml for the multiple sampling group and 224 ng.h/ml for the single sampling group. This picture remained constant for the other two JNJ compounds. The inter-animal variability with respect to plasma concentrations and the major PK parameters was low as observed from the multiple sampling groups.

2.2. Comparison of Plasma PK Parameters Following Administration Via the Jugular Or Saphenous Vein

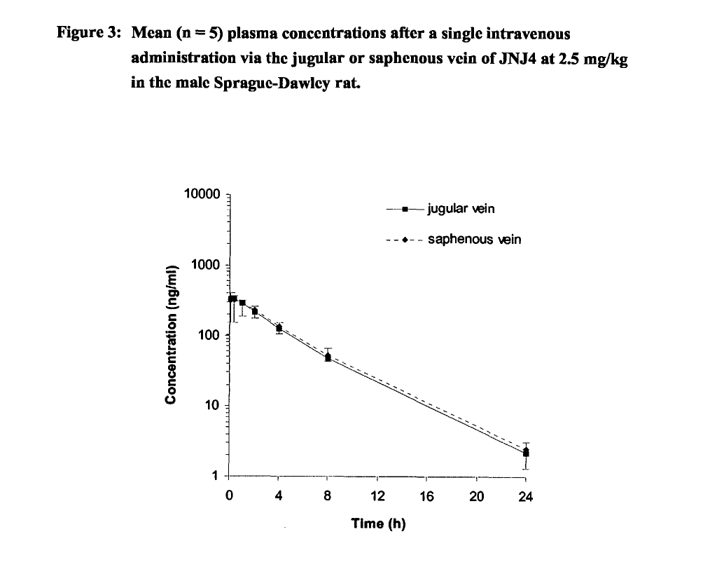

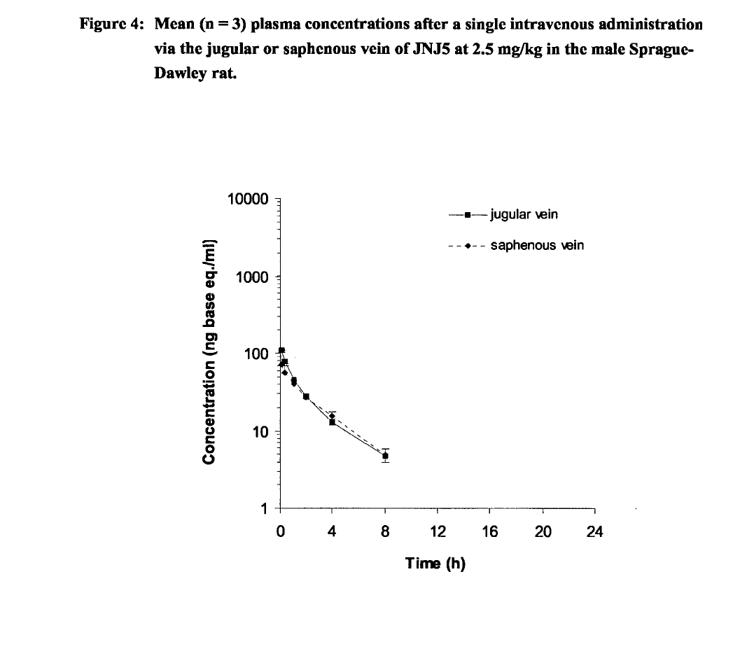

Eleven compounds (not all reported here) were dosed using the two routes. All compounds showed comparable results at the level of the plasma concentration time curves and the major PK parameters. As an example, the plasma concentration time profiles and basic PK parameters following a single iv administration at 2.5 mg/kg via the jugular vein cannula or directly into the saphenous vein of the compounds JNJ4, JNJ5 and JNJ6 are presented in tables 4-6 and graphically in FIGS. 3-5. As can be seen from the results both sampling techniques produced comparable plasma concentration time profiles and major PK parameters for the three compounds shown. Using JNJ 4 as an example, mean (n=5) plasma concentration time profiles of both administration routes showed a similar pattern (FIG. 3). Plasma concentrations declined monophasically with calculated half-lives (t_{1/2}) of 2.9 h for the jugular vein and 3.2 h for the saphenous vein administration (Table 4). Mean plasma clearances (Cl) were estimated at 1.7 l/h/kg for the jugular vein and 1.6 l/h/kg for the saphenous vein administration. The mean volume of distribution (Vdₛₛ) was estimated at 7.3 l/kg for the jugular and 7.5 l/kg for the saphenous vein administration. The calculated mean exposure (AUChd inf) for the jugular vein administration was 1445 ng.h/ml and for the saphenous vein 1512 ng.h/ml. As can be seen from FIG. 4 with JNJ5, plasma concentrations were only detectable until 8 hours post dose. However similar concentration time profiles and the corresponding PK parameters were calculated (Table 5).

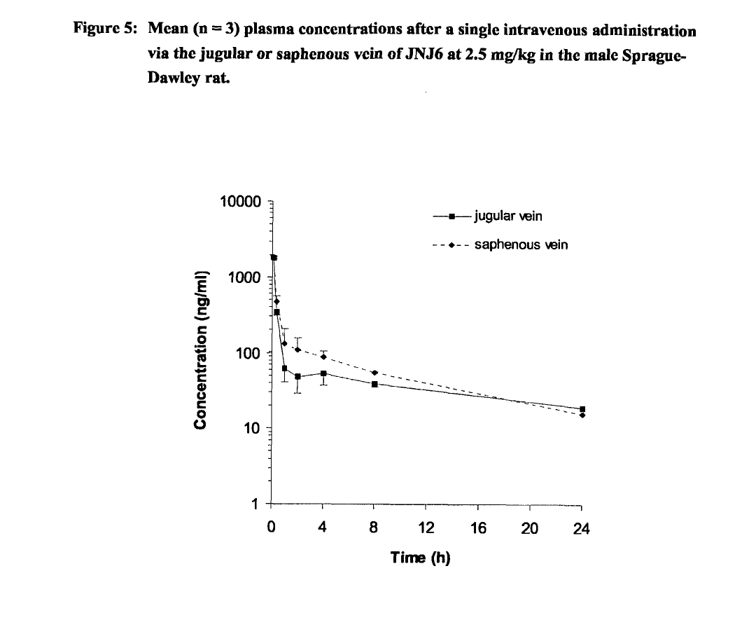

As can be seen from the graph the plasma concentrations of JNJ6 declined quite rapidly over the first hour with subsequent time points yielding low plasma levels (FIG. 5). The PK parameters from the two administration routes show comparable values for the plasma clearance and AUC (Area Under the Curve), but a larger difference in values for the terminal plasma half-life and the volume of distribution (Table 6). This is due to the small decreases in plasma concentrations between 2-24 h post dose. Overall, with respect to the rat, plasma half-lives values across this range are considered long and the volumes of distribution high. From other studies carried out with this compound (not reported here) the compound undergoes extensive tissue distribution to the major organs, confirming the high value of the volume of distribution observed here.

### 3. Discussion

3.1. Multiple Sampling Technique

The method of single blood sampling is one of several methods used for studying plasma PK in rats. The results derived from this method are obtained from multiple animals, sampled at different time points and pooled (the blood samples at each time point or the analysed data). The data analysis and calculation of the major PK parameters is then performed on the average plasma concentration time profile of the pooled data. This method of sampling does not allow the study of the variation in PK results between animals. Therefore a new sampling regimen involving multiple sampling in individual animals was investigated. Sufficient plasma samples were removed from each rat to construct a suitable concentration time profile, but without influencing the well being of the animals, namely at the level of the Hct. Alterations in the Hct indicates that the amount of blood cells has changed, which could influence the binding and distribution of many endogenous and exogenous substances and is thus an important factor in the PK of drugs. The Hct levels of rats were examined following blood removal at different volumes over the desired time period. The size of the blood sample had to be of sufficient volume for bioanalysis, i.e. compatible with the extraction procedure and subsequent analysis by LC-MS/MS. Sampling with a volume of 0.3 ml via the tail vein at seven different time points (t=7 and 20 min, 1, 2, 4, 8 and 24 h) from each animal (total volume 2.1 ml per day), appeared to have limited influence on the Hct value of the rat. Although there was a decrease in the % volume of Hct due to blood removal, the values of the last blood sample (37-42%) still ranged within the expected values for the healthy rat (36-48%).⁷ Sampling with a volume of 0.4 ml (total volume 2.8 ml), appeared to have more influence on the Hct value. The volume % Hct started to decrease significantly after the 2^{nd} blood sample and showed values below the expected values for the healthy rat at the last (t=24 h) blood sample (35-39%). These data show that blood removal with a total volume of 2.1 ml within 24 h has no dramatic effect on the Hct levels, whereas these values start to drop below expected healthy levels at volumes higher than 2.4 ml. However, it has to be mentioned that these levels were still borderline. To minimize the effect of the sampling volume on the Hct value, a total sampling volume of 2.1 ml per day and less was recommended for future studies. The removal of 0.3 ml blood from the tail vein at each time point allows sufficient samples (7) to be withdrawn from each animal to adequately describe the plasma concentration time curve and calculation of major PK parameters.

The comparison of the results of the PK study, carried out on the plasma concentration profiles of the shown compounds using the multiple and single sampling technique, allowed the comparison and subsequent validation of the new sampling method. The results illustrated that the sampling of seven small (0.3 ml) blood samples at defined time points from three individual animals gives comparable results to using three individual animals per time point, and therefore the multiple sampling technique can be used as an alternative method. The multiple sampling method has the advantage that multiple blood samples at different time points can be withdrawn from the same animal so that inter-animal variability can be examined. For the three compounds it was shown that the inter-animal variability was in fact low. Also it dramatically reduces the number of animals required to conduct a routine bioavailability study for one CE. The sampling method is robust and reproducible and has now been implemented in all bioavailability studies in the rat.

When using this sampling technique, ideally the tail vein should not be used as a route of iv administration of compounds due to the risk of contamination from the site of administration and sampling problems later on (due to over use and collapse of the veins). To overcome this aspect, in our Drug Discovery group compounds were routinely administered by injection via an indwelling jugular vein catheter. However, placing an indwelling catheter in the jugular vein under anaesthesia was time consuming (for surgery and recovery time) and caused some animal trauma. Therefore a new iv administration route, a direct injection via the saphenous vein in the awake rat, was investigated.

3.2. Saphenous Vein Administration

The saphenous vein is relatively small in relation to the jugular vein, however, the blood flow past the injection site should be of sufficient capacity to carry the compound into the systemic circulation. An advantage of this new technique is that no ligation of any blood vessels occurs as compared to the chronic implantation of a catheter, allowing the blood supply in the animal to remain unaltered. Another advantage is that it saves time as no surgery and recovery time is required. This in turn helps to speed up the throughput in the examination of NCEs in terms of the iv PK parameters. The present study was designed to investigate whether administration via the saphenous vein leads to a similar PK profile obtained using one of the classical administration techniques (via a jugular vein). Compounds with a diversity in chemical structure and solubility were used to show that this new technique could be used for many types of chemistry encompassing different disease area targets (5) and differing formulations (distilled water, 10 to 20% hydroxyl-β-cyclodextrin). After dosing 12 different compounds via the two different routes, a comparison of the obtained PK results has demonstrated that direct injection into the saphenous vein coupled with multiple sampling from the tail vein, produced similar plasma concentration time profiles and comparable PK results to those following jugular vein administration with the same method of blood sampling. For all compounds it was shown that administration via both iv routes led to some inter-individual variability of the plasma concentration levels but this was within the expected and acceptable range when working with animals.

3.3. Conclusion

The new techniques reduce the time required for preparative surgery and recovery (1-2 days), and allow the inter-animal comparison of major PK parameters as concentration time curves can be collected from each animal. The sampling of routine small blood samples does not have any major impact on the Hct of the rat and is compatible for the bioanalytical considerations. Finally, a reduction in the number of animals required for such a routine study is possible if compared to those using individual animals per time point and or sampling procedures involving anaesthesia.

In combination with the multiple-sampling technique, the new administration route via the saphenous vein will be used routinely for all future rat studies in Drug Discovery, where an iv administration is required.

TABLE 1
Basic pharmacokinetic parameters following a single
oral administration (10 mg/kg) of JNJ1 in the rat.

| | JNJ1 | | | | | |
|---|---|---|---|---|---|---|
| | | | | | | Single |
| | Multiple sampling | | | | | sampling |
| | Rat 1 | Rat 2 | Rat 3 | Mean | SD | Mean |
| Cₘₐₓ (ng/ml) | 81.7 | 68.7 | 47 | 63.6 | 17.5 | 41.4 |
| Tₘₐₓ (h) | 1 | 1 | 3 | 1 | 1.15 | 1 |
| t_{1/2} (h) | 2.31 | 1.72 | 2.04 | 2.02 | 0.30 | 1.92 |
| AUCₗₐₛₜ | 305 | 271 | 226 | 268 | 39.6 | 205 |
| (ng.h/ml) | | | | | | |
| AUC_{inf} | 342 | 288 | 251 | 294 | 46.0 | 224 |
| (ng.h/ml | | | | | | |

SD = standard deviation,
Cₘₐₓ = maximum plasma concentration,
Tₘₐₓ = time to reach Cₘₐₓ,
t_{1/2} = half life,
AUCₗₐₛₜ = observed exposure,
AUC_{inf} = extrapolated exposure.

TABLE 2
Basic pharmacokinetic parameters following a single oral
administration (5 mg/kg) of JNJ2 in the rat.

| | JNJ2 | | | | | |
|---|---|---|---|---|---|---|
| | | | | | | Single |
| | Multiple sampling | | | | | sampling |
| | Rat 1 | Rat 2 | Rat 3 | Mean | SD | Mean |
| Cₘₐₓ (ng/ml) | 135 | 96.3 | 108 | 113 | 19.8 | 155 |
| Tₘₐₓ (h) | 3 | 3 | 3 | 3 | 0 | 3 |
| t_{1/2} (h) | 6.81 | 4.89 | 5.65 | 5.75 | 0.97 | 4.93 |
| AUC ₗₐₛₜ | 1313 | 1125 | 866 | 1101 | 224 | 1380 |
| (ng.h/ml) | | | | | | |
| AUC_{inf} | 1438 | 1180 | 913 | 1175 | 263 | 1437 |
| (ng.h/ml) | | | | | | |

SD = standard deviation,
Cₘₐₓ = maximum plasma concentration,
Tₘₐₓ = time to reach Cₘₐₓ,
t_{1/2} = half life,
AUCₗₐₛₜ = observed exposure,
AUC_{inf} = extrapolated exposure.

TABLE 3
Basic pharmacokinetic parameters following a single oral
administration (2.5 mg/kg) of JNJ3 in the rat.

| | JNJ3 | | | | | |
|---|---|---|---|---|---|---|
| | | | | | | Single |
| | Multiple sampling | | | | | sampling |
| | Rat 1 | Rat 2 | Rat 3 | Mean | SD | Mean |
| Cₘₐₓ (ng/ml) | 106 | 132 | 137 | 125 | 16.6 | 149 |
| Tₘₐₓ (h) | 1 | 1 | 1 | 1 | 0 | 1 |
| t_{1/2} (h) | 9.77 | 5.88 | 4.6 | 6.8 | 2.7 | 9.12 |
| AUCₗₐₛₜ | 543 | 529 | 529 | 534 | 8 | 657 |
| (ng.h/ml) | | | | | | |
| AUC_{inf} | 623 | 839 | 748 | 737 | 108 | 753 |
| (ng.h/ml) | | | | | | |

SD = standard deviation,
Cₘₐₓ = maximum plasma concentration,
Tₘₐₓ = time to reach Cₘₐₓ,
t_{1/2} = half life,
AUCₗₐₛₜ = observed exposure,
AUC_{inf} = extrapolated exposure.

TABLE 4
Basic pharmacokinetic parameters after a single intravenous
administration via the saphenous or jugular vein of JNJ4
at 2.5 mg/kg in the male Sprague-Dawley rat.

| | JNJ4 | | | | | | |
|---|---|---|---|---|---|---|---|
| | Rat 1 | Rat 2 | Rat 3 | Rat 4 | Rat 5 | Mean | SD |
| | Saphenous vein administration | | | | | | |
| Cl (l/h/kg) | 1.6 | 2.2 | 1.4 | 1.6 | 1.4 | 1.6 | 0.3 |
| Vdₛₛ (l/kg) | 8.5 | 7.8 | 6.1 | 6.2 | 8.7 | 7.5 | 1.2 |
| t_{1/2} (h) | 3.7 | 2.4 | 2.9 | 2.7 | 4.4 | 3.2 | 0.8 |
| AUCₗₐₛₜ ng.h/ml) | 1447 | 995 | 1763 | 1621 | 1579 | 1481 | 294 |
| AUC_{inf} (ng.h/ml) | 1455 | 1100 | 1771 | 1636 | 1596 | 1512 | 256 |
| | Jugular vein administration | | | | | | |
| Cl (l/h/kg) | 1.5 | 1.6 | 2.2 | 1.6 | 1.6 | 1.7 | 0.3 |
| Vdₛₛ (l/kg) | 7.7 | 5.2 | 12.8 | 4.4 | 6.2 | 7.3 | 3.3 |
| t_{1/2} (h) | 3.6 | 2.2 | 4.1 | 1.8 | 2.7 | 2.9 | 1.0 |
| AUCₗₐₛₜ (ng.h/ml) | 1500 | 1617 | 1131 | 1758 | 1164 | 1434 | 277 |
| AUC_{inf} (ng.h/ml) | 1509 | 1622 | 1152 | 1769 | 1174 | 1445 | 274 |

SD = standard deviation,
Cl = plasma clearance,
Vdₛₛ = volume of distribution at steady-state,
t_{1/2} = half life,
AUCₗₐₛₜ = observed exposure,
AUC_{inf} = extrapolated exposure.

TABLE 5
Basic pharmacokinetic parameters after a single intravenous
administration via the saphenous or jugular vein of JNJ5 at
2.5 mg/kg in the male Sprague-Dawley rat.

| | JNJ5 | | | | |
|---|---|---|---|---|---|
| | Rat 1 | Rat 2 | Rat 3 | Mean | SD |
| | Saphenous vein administration | | | | |
| Cl (l/h/kg) | 15.0 | 12.5 | 14.1 | 13.8 | 1.3 |
| Vd_{z} (l/kg) | 53.5 | 37.7 | 53.9 | 48.4 | 9.2 |
| t_{1/2} (h) | 2.5 | 2.1 | 2.7 | 2.4 | 0.3 |
| AUCₗₐₛₜ ng.h/ml) | 152 | 187 | 155 | 165 | 19.4 |
| AUC_{inf} (ng.h/ml) | 167 | 200 | 178 | 182 | 16.8 |
| | Jugular vein administration | | | | |
| Cl (l/h/kg) | 12.0 | 13.3 | 12.8 | 12.7 | 0.7 |
| Vd_{z} (l/kg) | 46.0 | 40.6 | 38.5 | 41.7 | 3.9 |
| t_{1/2} (h) | 2.7 | 2.1 | 2.1 | 2.3 | 0.3 |
| AUCₗₐₛₜ (ng.h/ml) | 188 | 175 | 182 | 182 | 6.5 |
| AUC_{inf} (ng.h/ml) | 209 | 188 | 195 | 197 | 10.7 |

SD = standard deviation,
Cl = plasma clearance,
Vd_{z} = volume of distribution during the terminal phase,
t_{1/2} = half life,
AUCₗₐₛₜ = exposure,
AUC_{inf} = extrapolated exposure.

TABLE 6
Basic pharmacokinetic parameters after a single intravenous
administration via the saphenons or jugular vein of JNJ6 at
2.5 mg/kg in the male Sprague-Dawley rat.

| | JNJ6 | | | | |
|---|---|---|---|---|---|
| | Rat 1 | Rat 2 | Rat 3 | Mean | SD |
| | Saphenous vein administration | | | | |
| Cl (l/h/kg) | 1.4 | 1.1 | 1.6 | 1.4 | 0.2 |
| Vdₛₛ(l/kg) | 11.5 | 5.3 | 19.2 | 12.0 | 7.0 |
| t_{1/2} (h) | 9.1 | 4.3 | 11.7 | 8.4 | 3.8 |
| AUCₗₐₛₜ ng.h/ml) | 1665 | 2401 | 1317 | 1794 | 553 |
| AUC_{inf} (ng.h/ml) | 1837 | 2556 | 1606 | 2000 | 495 |
| | Jugular vein administration | | | | |
| Cl (l/h/kg) | 1.6 | 1.2 | 1.3 | 1.4 | 0.2 |
| Vdₛₛ (l/kg) | 34.5 | 17.3 | 15.9 | 22.6 | 10.3 |
| t_{1/2} (h) | 22.7 | 13.7 | 11.6 | 16.0 | 5.9 |
| AUCₗₐₛₜ (ng.h/ml) | 1083 | 1621 | 1563 | 1422 | 295 |
| AUC_{inf} (ng.h/ml) | 1582 | 2056 | 1905 | 1848 | 242 |

SD = standard deviation,
Cl =plasma clearance,
Vdₛₛ = volume of distribution at steady-state,
t_{1/2} = half life,
AUCₗₐₛₜ = exposure,
AUC_{inf} = extrapolated exposure.

1. Analytical method for the determination of a pharmakokinetic parameter in the awake rat, comprising the subsequent steps of:
(a) intravenous administration of a chemical entity through the saphenous vein;
(b) sampling the blood from the tail vein.

2. Analytical method according to claim 1, characterized in that in step (b) multiple blood samples are taken from the tail vein.

3. Analytical method according to any one of claims 1 to 2, characterized in that the method further comprises a step (c), incorporated after step (b) wherein the blood/plasma sample is analyzed using a bioanalytical technique to determine a pharmacokinetic parameter.
